(19) United States
(12) Patent Application Publication
(10) Pub. No.: US 2019/0164650 A1
(43) Pub. Date: May 30, 2019

(54) POLYDYNAMIC INTEGRATED HEALTHCARE INFORMATION PLATFORM

(71) Applicants: Jeffrey I. Schwartz, Franklin, MA (US); Jerry G. Blaivas, New York, NY (US); Stuart K.J. Smyth, Newmarket, NH (US); Anthony J. Sansone, Tyngsboro, MA (US)

(72) Inventors: Jeffrey I. Schwartz, Franklin, MA (US); Jerry G. Blaivas, New York, NY (US); Stuart K.J. Smyth, Newmarket, NH (US); Anthony J. Sansone, Tyngsboro, MA (US)

(73) Assignee: Symptelligence Medical Informatics, LLC, Tyngsboro, MA (US)

(21) Appl. No.: 15/827,877

(22) Filed: Nov. 30, 2017

(51) Int. Cl. G16H 80/00; G16H 50/20; G16H 10/60; G06Q 40/08

(52) U.S. Cl. CPC G16H 80/00; G16H 50/20; G16H 50/30; G06Q 40/08; G16H 10/60

(57) ABSTRACT

Methods, systems and computer readable media for polydynamic integrated healthcare information processing platforms are described.

### FIELD

Some implementations relate generally to medical information systems and, more particularly, to systems, methods and computer readable media for quantitative polydynamic integrated healthcare information processing platforms.

### BACKGROUND

With growing confines resulting from healthcare reform and other changes, physicians are often pressured to see more patients in less time for less reimbursement, which may force physicians to essentially treat patient populations by a rubric. One problem in clinical practice may be that truly individualized care often requires collecting and analyzing clinical information that a patient provides. Another problem is that data collected may also be on-deterministic making it difficult to perform the necessary analysis or to utilize the data to improve treatment of current and similar cases.

Due to the changing healthcare delivery environment, individualized treatment may be undermined by the often time consuming and cumbersome nature of collecting necessary clinical data manually using conventional techniques. This problem may be exacerbated by an enormous administrative burden that may become too expensive for the physician. As a result, sufficient data may not be collected, inaccurate, or inconsistent and patients may be treated empirically (e.g., by trial and error). This trial and error approach can lead to inaccurate diagnoses, improper treatment for patients, and ultimately a greater burden on resources (e.g., time, money, etc.) on one or more participants in the healthcare delivery environment.

A need may exist for an integrated system that can connect patients, health care providers, and payors in such a way that improves the computerized collection, provides quantitative analysis and reporting of patient generated healthcare data (PGHD), assists in making recommendations for diagnosis and treatment, and makes the healthcare delivery process more visible to the patient, without major disruption to some conventional information technology components being used in healthcare. Some implementations were conceived in light of the above-mentioned problems and limitations, among other things.

### SUMMARY

Some implementations can include a computerized integrated healthcare information platform that can include one or more processors coupled to a nontransitory computer readable medium having stored thereon instructions that when executed by the one or more processors, cause the one or more processors to perform operations. The operations can include providing an interface for receiving electronic patient generated healthcare data from one or more of a mobile application, a patient portal, and one or more active forms, wherein the electronic patient generated healthcare data corresponds to a patient.

The operations can also include determining quantitatively one or more scores based on the electronic patient generated healthcare data, wherein the one or more scores are stored in the nontransitory computer readable medium, and providing the patient generated healthcare data and the one or more scores to a physician portal. The operations can further include receiving electronic treatment instruction data and clinical pathway selection from the physician portal, and generating a clinical pathway for the patient based on the electronic treatment instruction data.

The operations can also include receiving additional electronic patient generated healthcare data during a course of treatment based on the clinical pathway, wherein the additional electronic patient generated healthcare data corresponds to a patient, and determining a patient treatment outcome based on the additional electronic patient generated healthcare data. The operations can further include continuing treatment according to the clinical pathway when the patient treatment outcome is within an acceptable range, and providing an alert to a physician via the physician portal when the patient treatment outcome is not within an acceptable range.

In some implementations, the operations can include providing an electronic alert message to at least one of the patient and the physician when a monitored parameter is not within a given range. The operations can further include providing an electronic alert message to one or more payors that precertification is not met when patient generated healthcare data does not correspond to one or more qualifications for treatment.

The operations can also include identifying whether the patient needs treatment based on the patient generated healthcare data, and routing the patient within the computerized integrated healthcare information platform to a treatment pathway based on identifying whether the patient needs treatment. The operations can further include automatically determining a phenotype for the patient based on the patient generated healthcare data.

Some implementations can include an application programming interface module configured to provide an interface between the computerized integrated healthcare information platform and one or more of an EMR/EHR system, a practice management system, and a research and analytics system.

The operations can further include providing an interface for generating a custom protocol or pathway for the patient, wherein the custom protocol or pathway is received via an interface on the physician portal and is stored in the nontransitory computer readable medium.

The operations can also include generating electronic billing data based on one or more of the electronic treatment instructions data, clinical pathway data, and the patient treatment outcome.

Some implementations can include a method for operating a computerized integrated healthcare information platform. The method can include defining clinical pathway requirements based on one or more of assessments, clinical metrics, a scoring system, a measurement system, and one or more of an electronic diary and electronic questionnaire for monitoring key pathway routing indicators. The method can also include converting clinical content including converting the assessments into standard models, determining a scoring model, and determining one or more metrics or thresholds. The method can further include importing content into the computerized integrated healthcare information platform, and configuring computerized integrated healthcare information platform components. The method can also include deploying a test clinic, and promoting clinical pathway content to a production system.

Importing content into the computerized integrated healthcare information platform can include one or more of: importing one or more assessments, importing one or more metrics or thresholds, creating one or more report models, and creating one or more pathway containers.

Configuring computerized integrated healthcare information platform components can include one or more of: adding pathway components to a physician portal, extending a reporting model for the physician portal, and importing one or more of assessments and diary components to a patient application.

Deploying a test clinic can include one or more of: creating a test clinic, testing clinical content associated with both patients and providers, and verifying assessment models, scoring and sub scoring algorithms, metrics, thresholds, and reports.

Promoting clinical pathway content to a production system can include one or more of deploying to a production system, adding new content to clinics, and deploying to one or more of a patient mobile application and a patient portal.

Some implementations can include a computer implemented method. The method can include providing an interface for receiving electronic patient generated healthcare data at a computerized integrated healthcare information platform from one or more of a mobile application, a patient portal, and one or more active forms, wherein the electronic patient generated healthcare data corresponds to a patient. The method can also include determining one or more scores based on the electronic patient generated healthcare data, and providing the patient generated healthcare data and the one or more scores to a physician portal.

The method can further include receiving electronic treatment instruction data and clinical pathway selection from the physician portal, and generating a clinical pathway for the patient based on the electronic treatment instruction data. The method can also include receiving additional electronic patient generated healthcare data during a course of treatment based on the clinical pathway, wherein the additional electronic patient generated healthcare data corresponds to a patient, and determining a patient treatment outcome based on the additional electronic patient generated healthcare data.

The method can also include continuing treatment according to the clinical pathway when the patient treatment outcome is within an acceptable range, and providing an alert to a physician via the physician portal when the patient treatment outcome is not within an acceptable range.

The method can further include providing an electronic alert message to at least one of the patient and the physician when a monitored parameter is not within a given range. The method can also include providing an electronic alert message to one or more payors that precertification is not met when patient generated healthcare data does not correspond to one or more qualifications for treatment.

The method can further include identifying whether the patient needs treatment based on the patient generated healthcare data, and routing the patient within the computerized integrated healthcare information platform to a treatment pathway based on identifying whether the patient needs treatment.

The method can also include automatically determining a phenotype for the patient based on the patient generated healthcare data.

The method can also include providing an interface for generating a custom protocol or pathway for the patient, wherein the custom protocol or pathway is received via an interface on the physician portal and is stored in a nontransitory computer readable medium.

### BRIEF DESCRIPTION OF THE DRAWINGS

FIG. 1 is a diagram of an example polydynamic integrated healthcare information processing platform environment in accordance with at least one implementation.

FIG. 2 is a flowchart of an example polydynamic integrated healthcare information processing method in accordance with at least one implementation.

FIG. 3 is a diagram of an example polydynamic integrated healthcare information processing platform in accordance with at least one implementation.

FIG. 4 is a flowchart of an example clinical pathway method in accordance with at least one implementation.

FIG. 5 is a diagram of an example polydynamic integrated healthcare information processing platform environment in accordance with at least one implementation.

FIG. 6 is a flowchart of an example polydynamic integrated healthcare information processing method in accordance with at least one implementation.

FIG. 7 is a diagram of an example computer system configured as a polydynamic integrated healthcare information processing platform in accordance with at least one implementation.

FIG. 8 is a diagram of an example user interface of a polydynamic integrated healthcare information processing platform in accordance with at least one implementation.

### DETAILED DESCRIPTION

Some implementations can include features that represent technical solutions to problems in, and improvements to, computing technology in the healthcare industry. For instance, the polydynamic integrated healthcare information processing platform (or simply “the platform”, as used herein) represents a significant improvement over conventional solutions in several areas such as quantitative clinical pathway support and patient phenotyping support, just to name two examples.

Some implementations can include improved quantitative clinical pathway support in which the platform provides quantitative data, tools, metrics and visualizations that permit accurate and precise measurement of patient symptoms, and patient generated health data (PGHD), and can provide concise in-context reports to healthcare providers and/or patients. This data can then be used to quantitatively compare patients to pathways, and in turn to symptoms, and accurately generating and record a mapping between PGHD, symptoms, and pathways. This represents a major improvement over some conventional systems that may be based on qualitative methods which may not be precise and may result in many cases of unneeded or unnecessary care. The quantitative approach of the disclosed platform can help do the following: reduce the cost of healthcare, improve the accuracy of healthcare, and/or improve the patient experience and outcome.

The platform can use electronic questionnaires and diaries to collect electronic representations of patient generated healthcare data, match them to scoring rubrics that contain both total scores (gross indicators of overall severity) and sub scores that highlight specific indicators of underlying patient conditions. Because patient data is collected and scored consistently, patient data can be accurately measured over time to diagnose and track patient conditions, and also responsiveness to treatments.

The platform also represents an improvement over some previous solutions in that the platform applies the data and algorithms to patient phenotyping (the ability to identify and segregate patients into potential treatment groups (pathways) via remote methods) so that phenotyping can be made more accurate and potentially performed prior to an initial office visit. The patient data gathered can be measured via algorithms in the platform and presented via visualizations of the platform to provide support to clinicians and navigators to help more effectively and accurately identify patients that need treatment in general populations, and to sort them into the most effective treatment pathway earlier. This technical improvement can result in improvements to outcomes and can help reduce costs associated with quality patient care.

Further, by combining the platform's individual patient generated health data collection, quantitative analysis and scoring methodology with patient specific clinical pathways, treatments, outcomes, number of visits, visit times and resources, a health organization can more effectively perform the following:

(1) Predictive analyses of costs, resource capacity, utilization rates, and projected revenue providing an effective tool to manage and, more importantly, improve organizational performance both clinically and operationally. This can be especially vital as the reimbursement model moves away from fee-for-service to outcome based models.

(2) Risk stratification of patient population by probability of severity of symptoms for management of patient intake triage and prioritization. Reporting will allow the filtering of data to optimize resources while providing the right services to the appropriate patient and the right time.

(3) Chronic care plan effectiveness and compliance for patients under chronic care plans. Quantitative reporting of patient data enables remote monitoring of patient conditions and responses to chronic care plans so that corrective actions can be detected and managed efficiently.

It will be appreciated that although some examples described herein may be directed to urinary conditions, the platform can be applied to any suitable medical condition.

Some implementations include applying algorithms that generate scores (and sub-scores) using PGHD as input and using these scores to assist in clinical pathway selection, evaluation, and visualization.

Clinical pathways can be made “smart” by using the platform to send better qualified patients through a given pathway. Some implementations can include using machine learning to evolve a pathway model and enable the platform to even better qualified patients through given pathways.

Some implementations can help quantify a patient's condition and help management prioritize access to healthcare resources based on quantitative condition measures, which is in contrast to some conventional systems that may rely on anecdotal and/or subjective information gathering techniques.

Some implementations can provide an improved patient measurement and tracking method and system. A body of knowledge in a specific clinical area can be used to generate standardized testing, surveys, diary questions, etc. to obtain patient generated healthcare data. Normalized quantitative values (e.g., scores and subscores) can be generated from the PGHD.

Patient progress can be tracked over time using the normalized quantitative data. Data from multiple patients can be combined (with permission of respective patients to use their data) to make inferences about populations and phenotypes.

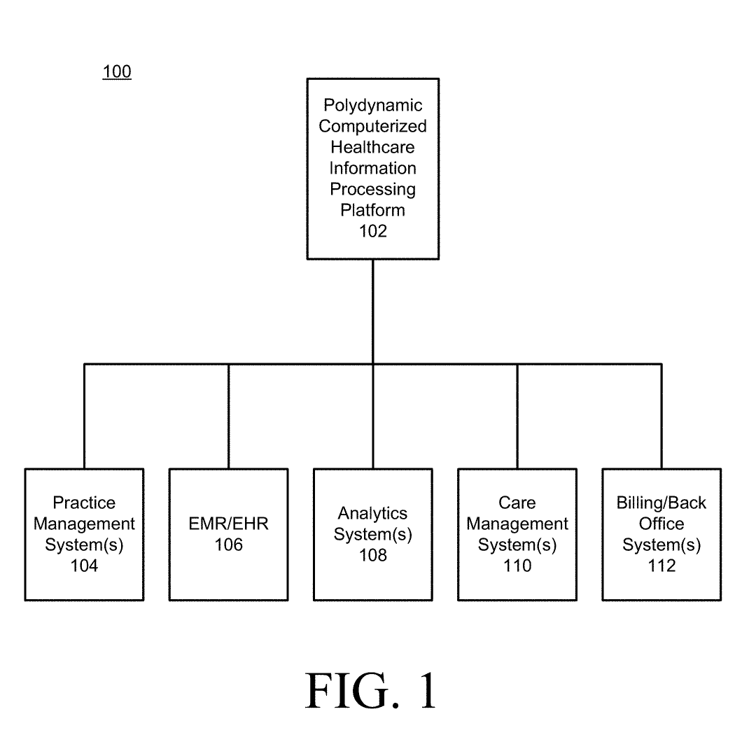

FIG. 1 is a diagram of an example polydynamic integrated healthcare information processing platform environment 100 in accordance with at least one implementation. The environment 100 can include a polydynamic computerized healthcare information processing platform 102, which can be coupled to various other systems such as one or more practice management systems 104, one or more EMR/HER systems 106, one or more analytics systems 108, one or more care management systems 110, and/or one or more billing/back office systems 112. FIG. 1 illustrates the extent to which a platform as disclosed herein can integrate with existing healthcare data systems.

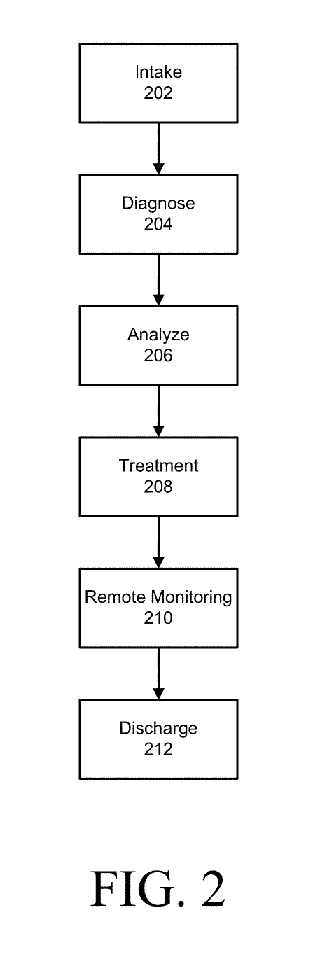

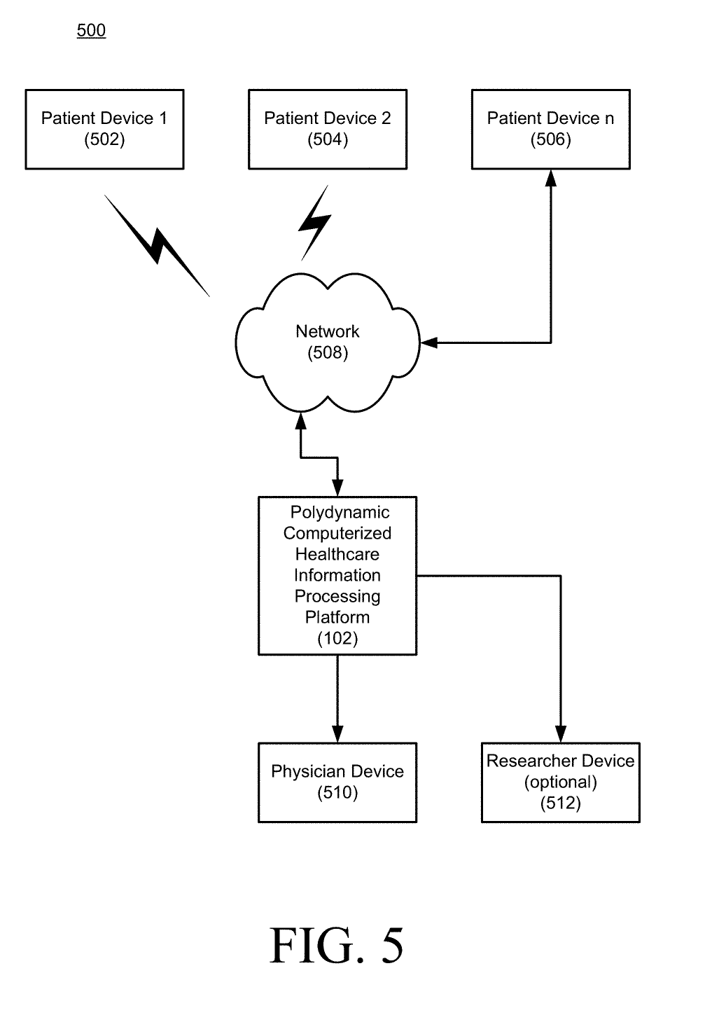

FIG. 2 is a flowchart of an example polydynamic integrated healthcare information processing method in accordance with at least one implementation. Processing begins at 202, where patient intake is performed. An implementation of the platform can provide improvements to patient intake by having a patient using a mobile application or other software to collect patient generated healthcare data (PGHD) prior to the first visit. Accordingly, at intake time, significant data about the patient's condition may be available from the mobile application or website in the form of diary entries (e.g., symptoms, quantities, time of day of events, etc.), questionnaire responses, etc. This data may have been preprocessed by the mobile application or website, or may be processed during intake, to generate a clinical profile of the patient to be made available for use by the healthcare provider, as discussed below in connection with FIG. 5. Processing continues to 204.

At 204, an initial diagnosis hypothesis or suggestion can be automatically generated by the platform using the PGHD and/or scores/sub-scores. This diagnosis suggestion can be used as a guide for the healthcare provider. Processing continues to 206.

At 206, an analysis of the PGHD, score/sub-scores, and/or diagnosis can be performed in order to place the patient into a clinical pathway or develop a treatment plan for the patient. Processing continues to 208.

At 208, treatment can be ordered and started. The platform can maintain an electronic record of treatment orders. Processing continues to 2210.

At 210, patient progress is remotely monitored via the mobile application (or website/application). Updated PGHD can be updated during the treatment process and used to determine what, if any, effect the treatment is having on the patient's symptoms. If the patient's progress is satisfactory (e.g., PGHD parameters and/or scores/sub-scores within threshold values) the treatment can continue until a termination condition is reached (e.g., treatment is only prescribed for a given time period, patient symptoms dissipate to level where treatment is no longer needed, etc.). If the patient's progress is unsatisfactory (e.g., PGHD parameters and/or scores/sub-scores exceed threshold values), the patient and/or healthcare provided can be provided with an electronic alert and the healthcare provider may evaluate whether to order a change in treatment regimen. Processing continues to 212.

At 212, a patient can be discharged from treatment/monitoring when the patient improves to a level where treatment/monitoring is no longer needed or for other conditions (e.g., symptoms no longer meet precertification criteria for treatment, etc.).

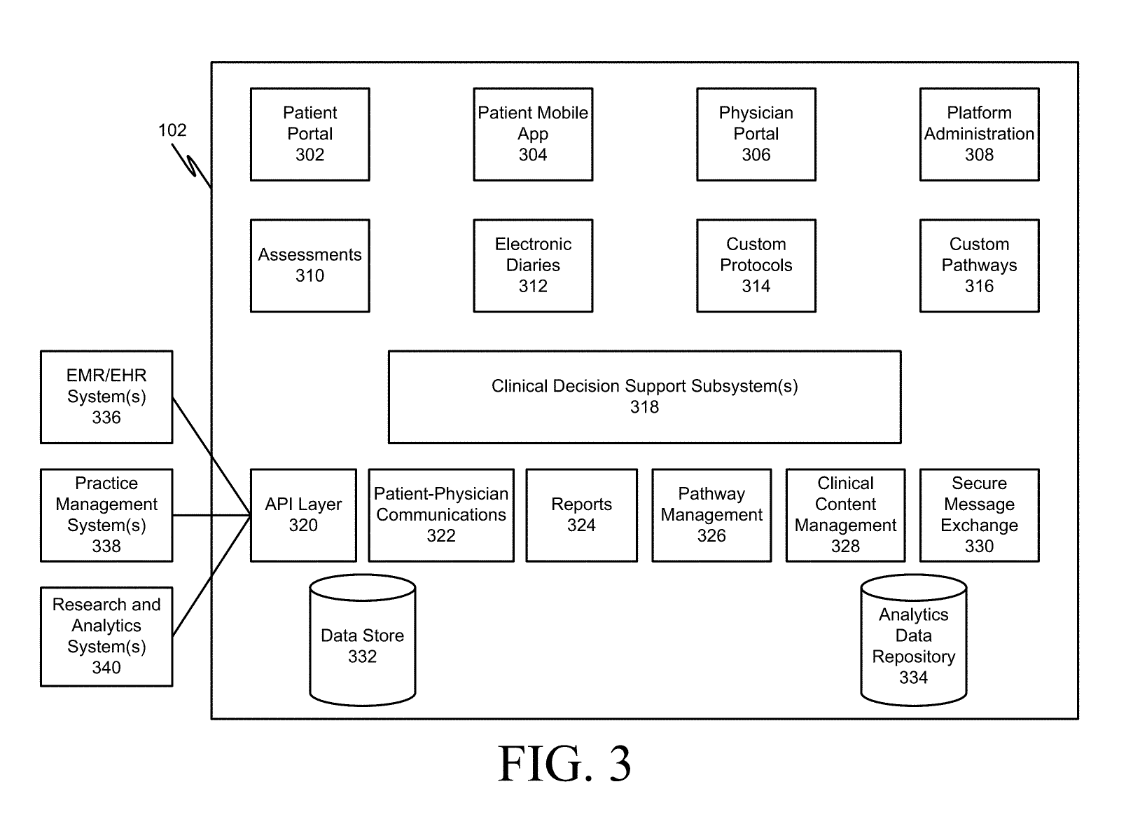

FIG. 3 is a diagram of an example polydynamic integrated healthcare information processing platform 102 in accordance with at least one implementation. The platform 102 can include a patient portal 302 that can be accessed by the patient via a patient mobile application 304, a web browser, a stand-alone application on a computer, or the like. The patient portal 302 can include a patient portal such as that described in International Patent Application No. PCT/US15/31450, filed May 18, 2015, which is incorporated herein by reference in its entirety.

The platform can also include a physician portal 306 such as that described in PCT/US15/31450. The platform 102 can include a platform administration module 308, which can be used to administer the platform 102.

The platform 102 can include one or more assessments 310 and electronic diaries 312, which can be used to gather PGHD from patients via the patient portal 302 and/or patient mobile application 304. The platform 102 can also include custom protocols 314 and customer pathways 316 that can be selected based on the PGHD and scores/sub-scores of a respective patient.

The platform 102 can include clinical decision support subsystems 318 based on proprietary techniques.

The platform 102 can include one or more application programming interfaces (APIs) in an API layer 320. The API layer 320 can be configured to couple the platform 102 to one or more external systems such as one or more EMR/EHR systems 336, one or more practice management systems 338, one or more research or analytics systems 340, etc. PGHD can be quantified as described herein and can be provided to one or more research or analytics systems 340 to be used for analytics, process improvement and clinical research support.

The platform 102 includes support for patient-physician communications 322, which can include providing a secure message exchange system 330 for patients to communicate with physicians or other healthcare providers. Communications between patients and physicians can also be logged by the platform 102 and stored (e.g., in data store 332) for record keeping, quality assurance, etc.

The platform 102 includes a reports module 324 configured to generate reports for patients, healthcare providers, payors, researchers, and/or platform administrators. The platform 102 also includes a pathway management sub-system 326 that is configured to manage the pathways that patients are placed into based on PGHD and scores/sub-scores of respective patients the pathway management subsystems can include proprietary techniques.

The platform 102 includes a clinical content management module 328. The clinical content management module 328 is configured to manage clinical content such as assessments, clinical metrics, scoring system, measurement system, electronic diary, electronic questionnaires, etc. Clinical content management will allow the platform to be able to support and quickly deploy new solutions to address new markets or disease states.

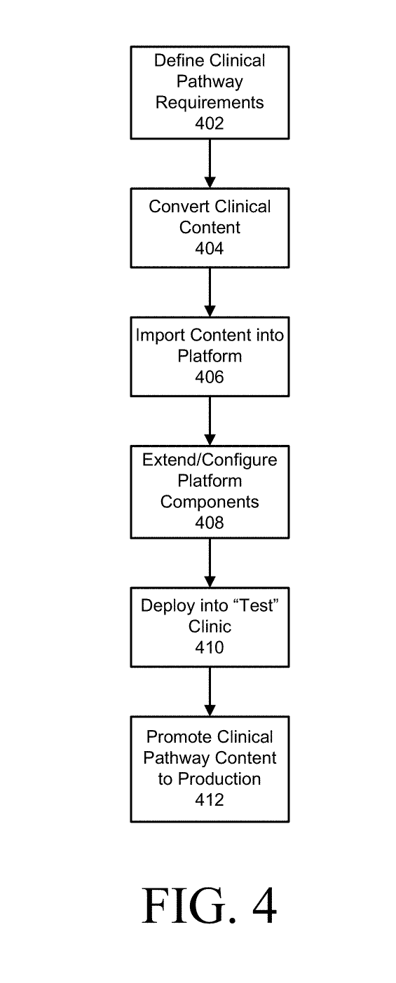

FIG. 4 is a flowchart of an example clinical pathway method in accordance with at least one implementation. Processing begins at 402, where clinical pathway requirements are defined. A clinical pathway is a methodology to help determine the most effective way to treat a patient with the least possible steps. A treatment plan is based on the pathway results and corresponds to the condition of the patient (e.g., a chronic condition). This invention provides for more granular determination of pathway based on phenotyping of individuals based on quantitative algorithms. Patient phenotyping from scoring algorithms. Processing continues to 404.

At 404, clinical content is converted. For example, accessing the clinical content and providing the clinical content through the portal to both physicians and patients, collecting the data, scoring the collected data against the metrics, and then reporting the results. Processing continues to 406.

At 406, converted clinical content is imported into the platform. Processing continues to 408.

At 408, platform components are extended/configured. For example, creating and deploying additional content, modifying and/or extending existing content. Processing continues to 410.

At 410, the pathway is deployed into a “test” clinic. Processing continues to 412.

At 412, clinical pathway content is promoted to production status. For example, clinical pathway content once vetted by qualified clinical personnel can be promoted to production within the system.

FIG. 5 is a diagram of an example polydynamic integrated healthcare information processing platform environment 500 in accordance with at least one implementation. In particular, the environment 500 can include a polydynamic integrated healthcare information processing platform 102. A plurality of user devices (504-508) can communicate with the polydynamic integrated healthcare information processing platform 102 via network 510. The polydynamic integrated healthcare information processing platform 102 can provide PGHD from one or more patients to a physician device 512 and, optionally, to a researcher device 514.

The patient devices (504-508) can include a desktop computer, laptop computer, tablet computer, wireless device, tablet computer, wearable computer, electronic book reader, media player and/or the like. The network 510 can include a wired or wireless network (e.g., a WiFi network, the Internet, etc.). For example, one or more patient devices (e.g., 504 and 506) can communicate wirelessly with the network 510, while other patient devices (e.g., 508) can communicate via a wired interface.

In operation, using an implementation of the platform as described herein, a patient can be instructed to download an implementation of the disclosed mobile application (or use a web-based version of the application) to his/her respective device (e.g., 504-508) and track his/her own symptoms (or health behavior) for a given period of time (e.g., a 24-hour period) before the patient even arrives at the physician's office. The software intelligently creates a clinical summary that can be seamlessly incorporated in the electronic medical record (EMR) of that patient. When the physician (or other healthcare provider) sees the patient for the first time, the basic clinical overview of the patient has been generated and discussion between the doctor and patient can focus on filling in specifics such as clinical pathway identification and treatment options. This is in contrast to conventional methods, which may require a healthcare provider to collect and sift through an enormous amount of clinical information to pinpoint symptoms and causes. With an implementation of the platform described herein, at the first visit, the healthcare provider has the necessary clinical information he/she would hope to typically collect in the first two patient visits under traditional approaches.

The current healthcare delivery model may include a physician collecting a general history in a limited time period and then advising a patient to try a medication and return depending on its efficacy. With an implementation of the disclosed platform, a doctor can begin with an individualized summary generated by the system, then spend time answering the patient's questions, and identifying and directly treating the underlying cause of the pathology via a pathway and/or treatment. This system can save time for the physician, reduce or eliminate the cost and administrative burden of collecting and transcribing clinical data, and most importantly, provide the patient with an individualized, targeted, and correct diagnosis and treatment pathway where indicated. An implementation can possibly save the medical system the costs of multiple physician visits, improper prescriptions, and worse, unnecessary treatments and/or surgeries. Through the disclosed platform, patients become part of the solution, helping doctors save time and money while ultimately making more informed clinical decisions that, in turn, saves the healthcare system money while helping provide better outcomes.

An implementation can have similar appeal for academic institutions, pharmaceutical companies, and any physician interested in clinical research. Collecting clinical data often requires time, motivated patients, and a staff dedicated to gathering and amassing this information. In fact, most clinical trials must offer patients either financial incentives or the promise of a unique chance to try a new drug for their condition. Beyond this cost, pharmaceutical companies or any institution running a trial must employ recruiters to enroll patients and subsequently must pay research assistants to transcribe the data from the patients into a system that can be queried. The use of an implementation of the disclosed platform system for trials and other clinical research can significantly reduce or eliminate these burdens. All inclusion and exclusion criteria can be programmed within the mobile application so the cost of recruiters drops immensely (if not entirely). Further, the mobile application software/portal system accumulates data seamlessly within a database that can be pre-configured in the desired format for research queries.

Some implementations can include a Precertification Model for ACO's. For example, the disclosed platform can be configured to work with global healthcare systems, such as fee for service basis, but can also be configured to operate in a pay for performance model (e.g., an ACO Model which is rapidly advancing in the USA).

The incorporation of an implementation of the disclosed platform system into a healthcare system can be beneficial for all stakeholders (e.g., patients, physicians, ACOs, and/or payors, etc.). The healthcare system may be seeking for cost control. Typically surgeries, and now some other procedures, may require the physician to provide evidence that the treatment is appropriate for the patient. The burden may be on the physician or on the ACO itself. The payor (be it insurance companies, the government, or pharmacy benefit managers) will mandate that doctors prove treatment is necessary, prudent and appropriate if the doctor wants to be reimbursed for treating a particular symptom or condition. The disclosed platform can be configured to serve as a precertification tool and can help transfer the information gathering task of precertification from the physician to the patient. The patient can record his/her symptoms using validated clinical tools to provide parameters that can categorize patients' symptoms. The motivation for the payor is clear: empirical treatment can possibly lead to more patient visits, prescription of drugs for a condition a patient doesn't have and even unnecessary surgeries. Mandating the precertification tool may make fiscal sense. Physicians may benefit from the time and money they will save along with the more nuanced care they will be able to provide. Patients, in turn, will get individualized care and start to better understand their own symptoms.

Some implementations can include a platform that connects to mobile applications or web-based applications for taking medical history, record keeping and informatics that can provide symptom specific questionnaires and diaries. Data can be entered on an application (e.g., on mobile device, tablet, laptop or desktop computer or the like) by the patient. Some implementations can include patients entering medical history or other medical related information themselves.

Some implementations do not require a connection to the internet. For example, a mobile application (or “app”) can summarize and categorize data for clinical use. In some implementations, the patient data is available for one or more of a personal electronic medical record (EMR), medical records (PDF or XML), diagnosis & treatment, monitoring outcomes, monitoring/preventing complications and adverse events and/or research.

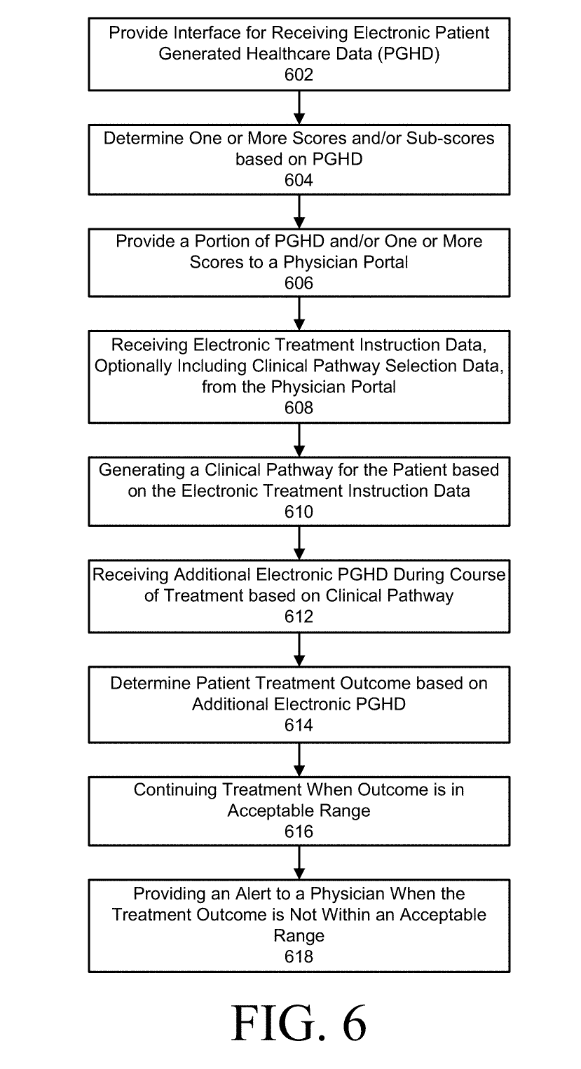

FIG. 6 is a flowchart of an example polydynamic integrated healthcare information processing method in accordance with at least one implementation. Processing begins at 602, where a platform interface is provided that is configured to receive PGHD from various sources, including, but not limited to, a mobile application (e.g., 304), a patient portal (e.g., 302), and/or one or more active forms. The platform receives PGHD at the interface. Processing continues to 604.

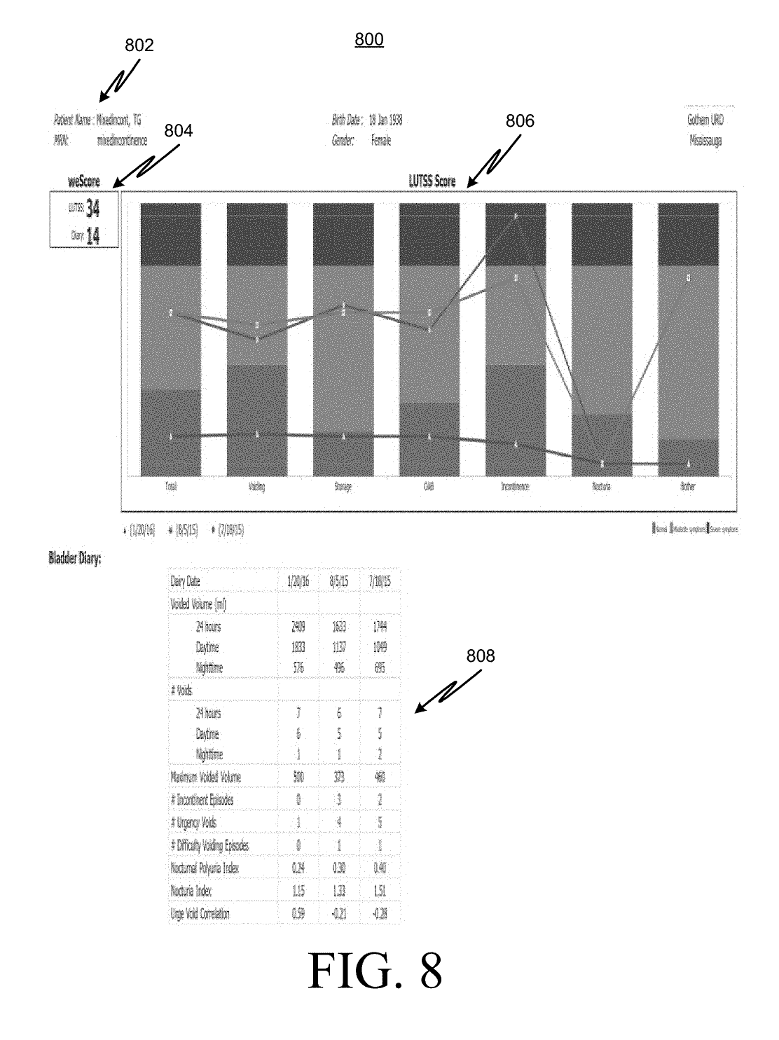

At 604, one or more scores (or sub-scores) is determined based on the PGHD corresponding to a patient received in 602. For example, in a urology setting, the scores/sub-scores can include one or more of a LUTSS score, a diary score, a composite score, a voiding score, a storage score, an OAB score, an incontinence score, a nocturia score and a bother score (as shown in FIG. 8). Processing continues to 606.

At 606, the PGHD and the one or more scores/sub-scores are provided to a physician portal (e.g., 306). A physician or other health care provider can then view the received PGHD and the one or more scores/sub-scores. Processing continues to 608.

At 608, electronic treatment instruction data, which can optionally include a clinical pathway selection, is received from the physician portal (or other system). Processing continues to 610.

At 610, a clinical pathway is generated for the patient based on the received electronic treatment instruction data and/or clinical pathway selection. Processing continues to 612.

At 612, receiving additional electronic PGHD during a course of treatment of the patient. The additional electronic PGHD corresponding to the patient. The additional electronic PGHD can include electronic/computerized assessment data, questionnaire responses, electronic medical diary entries, etc. Processing continues to 614.

At 614, an outcome or intermediate outcome is determined based on the additional electronic PGHD. For example, subsequent score/sub-scores can be generated using the additional electronic PGHD and those subsequent score/sub-scores can be used alone or in conjunction with earlier scores/sub-scores to determine an outcome or intermediate outcome of treatment being carried out according to the electronic treatment instruction data received at 608. Processing continues to 616.

At 616, treatment according to the electronic treatment instruction data received at 608 is continued when outcome or intermediate outcome is in an acceptable range. For example, if the scores/subscores show improvement or are within an acceptable range, then treatment may be continued as directed by a physician or other health care provider. The outcome or intermediate outcome data can be transmitted to the physician portal, patient portal, mobile app, etc. If the outcome or intermediate outcome is acceptable has reached a level that indicates no further treatment may be required, treatment could be terminated by a physician or health care provider via updated electronic treatment instruction data received from the physician portal or other system indicating to stop treatment in an appropriate manner. Processing continues to 618.

At 618, an electronic alert message may be provided by the platform to a physician, health care provider and/or the patient when an outcome or intermediate outcome of the treatment is not within acceptable range. For example, if one or more of the scores/sub-scores based on the additional electronic PGHD show a decline or worsening of symptoms (or show an improvement in symptoms that is either too small or too slow), the patient and/or the patient's physician or health care provider can receive an electronic alert message from the platform.

It will be appreciated that one or more of 602-618 can be repeated. For example during the course of treatment, 612-618 may be repeated in whole or in part in order to perform monitoring of the patient during treatment.

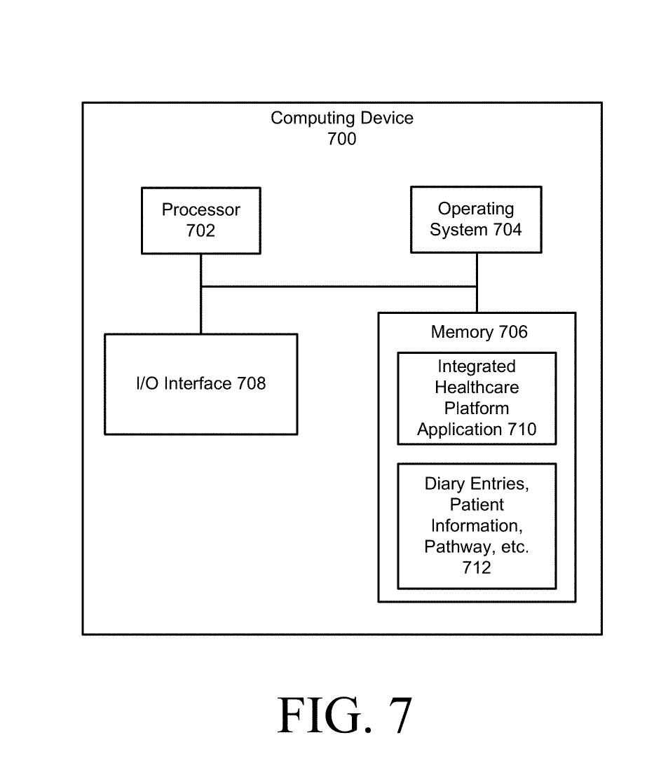

FIG. 7 is an example computer system 700 for a polydynamic integrated healthcare information processing platform in accordance with at least one implementation. The server device 700 includes a processor 702, an operating system 704, a memory 706 and an I/O interface 708. The memory 706 can include one or more polydynamic integrated healthcare information processing platform applications 710 and polydynamic integrated healthcare information processing platform records 712.

In operation, the processor 702 may execute the application 710 stored in the memory 706. The application 710 can include software instructions that, when executed by the processor, cause the processor to perform operations for a computerized medical informatics system in accordance with the present disclosure (e.g., performing one or more of steps 202-212, 402-412, and/or 602-612 described herein). The application program 710 can operate in conjunction with the medical data and/or patient information records 712 and the operating system 704.

FIG. 8 is a diagram of an example computer graphical user interface 800 of a polydynamic integrated healthcare information processing platform in accordance with at least one implementation. The computer GUI 800 includes patient information 802 (e.g., name, date of birth, gender, primary diagnosis, health care provider, physician name, etc.

The GUI 800 also includes score summary information 804. In this example, the score summary information includes a LUTSS (lower urinary tract symptom score) of 34 and a diary score of 14. It will be appreciated that these are example scores shown for illustration purposes. Implementations are not limited to these scores or to urology.

The GUI 800 also includes a graphical element 806 to visualize tracking total score/sub-score over time. For example, the element 806 includes columns for total score, voiding score, storage score, overactive bladder (OAB) score, incontinence score, nocturia score, and bother score. Within the columns of element 806 data points for each score for given set of dates (e.g., Jan. 20, 2016, Aug. 5, 2015, and Jul. 19, 2015) are shown and graphed. Ranges within each column show normal (green), moderate symptoms (orange), and sever symptoms (red).

The GUI 800 also includes electronic medical diary information 808 for one or more dates. For example, the diary information 808 includes diary date, and voided volume measurements/estimates for 24 hours, daytime and night time. The diary information 808 also includes a record of number of voids for the past 24 hours, daytime and nighttime. The diary information 808 also includes a maximum voided volume measurement or estimate, a record of the number of incontinence episodes, a record of the number of urgency voids, a record of the number of difficult voiding episodes, a nocturnal polyuria index value, a nocturia index value, and an urge void correlation. It will be appreciated that the electronic medical diary information 808 is shown for illustration purposes and that implementations are not limited to the information shown or to urological electronic medical diary information.

It will be appreciated that the modules, processes, systems, and sections described above can be implemented in hardware, hardware programmed by software, software instructions stored on a nontransitory computer readable medium or a combination of the above. A system as described above, for example, can include a processor configured to execute a sequence of programmed instructions stored on a nontransitory computer readable medium. For example, the processor can include, but not be limited to, a personal computer or workstation or other such computing system that includes a processor, microprocessor, microcontroller device, or is comprised of control logic including integrated circuits such as, for example, an Application Specific Integrated Circuit (ASIC). The instructions can be compiled from source code instructions provided in accordance with a programming language such as Java, C, C++, C#.net, assembly or the like. The instructions can also comprise code and data objects provided in accordance with, for example, the Visual Basic™ language, or another structured or object-oriented programming language. The sequence of programmed instructions, or programmable logic device configuration software, and data associated therewith can be stored in a nontransitory computer-readable medium such as a computer memory or storage device which may be any suitable memory apparatus, such as, but not limited to ROM, PROM, EEPROM, RAM, flash memory, disk drive and the like.

Furthermore, the modules, processes systems, and sections can be implemented as a single processor or as a distributed processor. Further, it should be appreciated that the steps mentioned above may be performed on a single or distributed processor (single and/or multi-core, or cloud computing system). Also, the processes, system components, modules, and sub-modules described in the various figures of and for embodiments above may be distributed across multiple computers or systems or may be co-located in a single processor or system. Example structural embodiment alternatives suitable for implementing the modules, sections, systems, means, or processes described herein are provided below.

The modules, processors or systems described above can be implemented as a programmed general purpose computer, an electronic device programmed with microcode, a hard-wired analog logic circuit, software stored on a computer-readable medium or signal, an optical computing device, a networked system of electronic and/or optical devices, a special purpose computing device, an integrated circuit device, a semiconductor chip, and/or a software module or object stored on a computer-readable medium or signal, for example.

Embodiments of the method and system (or their sub-components or modules), may be implemented on a general-purpose computer, a special-purpose computer, a programmed microprocessor or microcontroller and peripheral integrated circuit element, an ASIC or other integrated circuit, a digital signal processor, a hardwired electronic or logic circuit such as a discrete element circuit, a programmed logic circuit such as a PLD, PLA, FPGA, PAL, or the like. In general, any processor capable of implementing the functions or steps described herein can be used to implement embodiments of the method, system, or a computer program product (software program stored on a nontransitory computer readable medium).

Furthermore, embodiments of the disclosed method, system, and computer program product (or software instructions stored on a nontransitory computer readable medium) may be readily implemented, fully or partially, in software using, for example, object or object-oriented software development environments that provide portable source code that can be used on a variety of computer platforms. Alternatively, embodiments of the disclosed method, system, and computer program product can be implemented partially or fully in hardware using, for example, standard logic circuits or a VLSI design. Other hardware or software can be used to implement embodiments depending on the speed and/or efficiency requirements of the systems, the particular function, and/or particular software or hardware system, microprocessor, or microcomputer being utilized. Embodiments of the method, system, and computer program product can be implemented in hardware and/or software using any known or later developed systems or structures, devices and/or software by those of ordinary skill in the applicable art from the function description provided herein and with a general basic knowledge of the software engineering and medical informatics arts.

Moreover, embodiments of the disclosed method, system, and computer readable media (or computer program product) can be implemented in software executed on a programmed general purpose computer, a special purpose computer, a microprocessor, or the like.

It is, therefore, apparent that there is provided, in accordance with the various embodiments disclosed herein, systems, methods and computer readable media for polydynamic integrated healthcare information processing platforms.

While the disclosed subject matter has been described in conjunction with a number of implementations, it is evident that many alternatives, modifications and variations would be, or are, apparent to those of ordinary skill in the applicable arts. Accordingly, Applicants intend to embrace all such alternatives, modifications, equivalents and variations that are within the spirit and scope of the disclosed subject matter.

What is claimed is:

1. A computerized integrated healthcare information platform comprising:
one or more processors coupled to a nontransitory computer readable medium having stored thereon instructions that when executed by the one or more processors, cause the one or more processors to perform operations including:
providing an interface for receiving electronic patient generated healthcare data from one or more of a mobile application, a patient portal, and one or more active forms, wherein the electronic patient generated healthcare data corresponds to a patient;
determining one or more scores based on the electronic patient generated healthcare data, wherein the one or more scores are stored in the nontransitory computer readable medium;
providing the patient generated healthcare data and the one or more scores to a physician portal;
receiving electronic treatment instruction data and clinical pathway selection from the physician portal;
generating a clinical pathway for the patient based on the electronic treatment instruction data;
receiving additional electronic patient generated healthcare data during a course of treatment based on the clinical pathway, wherein the additional electronic patient generated healthcare data corresponds to a patient;
determining a patient treatment outcome based on the additional electronic patient generated healthcare data;
continuing treatment according to the clinical pathway when the patient treatment outcome is within an acceptable range; and
providing an alert to a physician via the physician portal when the patient treatment outcome is not within an acceptable range.

2. The computerized integrated healthcare information platform of claim 1, further comprising providing an electronic alert message to at least one of the patient and the physician when a monitored parameter is not within a given range.

3. The computerized integrated healthcare information platform of claim 1, further comprising providing an electronic alert message to one or more payors that precertification is not met when patient generated healthcare data does not correspond to one or more qualifications for treatment.

4. The computerized integrated healthcare information platform of claim 1, wherein the operations further comprise:
identifying whether the patient needs treatment based on the patient generated healthcare data; and
routing the patient within the computerized integrated healthcare information platform to a treatment pathway based on identifying whether the patient needs treatment.

5. The computerized integrated healthcare information platform of claim 1, wherein the operations further comprise: automatically determining a phenotype for the patient based on the patient generated healthcare data.

6. The computerized integrated healthcare information platform of claim 1, further comprising an application programming interface module configured to provide an interface between the computerized integrated healthcare information platform and one or more of an EMR/EHR system, a practice management system and a research and analytics system.

7. The computerized integrated healthcare information platform of claim 1, wherein the instructions further cause the one or more processors to:
provide an interface for generating a custom protocol or pathway for the patient, wherein the custom protocol or pathway is received via an interface on the physician portal and is stored in the nontransitory computer readable medium.

8. The computerized integrated healthcare information platform of claim 1, wherein the instructions further cause the one or more processors to:
generate electronic billing data based on one or more of the electronic treatment instructions data, clinical pathway data, and the patient treatment outcome.

9. A method for operating a computerized integrated healthcare information platform, the method comprising:
defining clinical pathway requirements based on one or more of assessments, clinical metrics, a scoring system, a measurement system, and one or more of an electronic diary and electronic questionnaire for monitoring key pathway routing indicators;
converting clinical content including converting the assessments into standard models, determining a scoring model, and determining one or more metrics or thresholds;
importing content into the computerized integrated healthcare information platform;
configuring computerized integrated healthcare information platform components;
deploying a test clinic; and
promoting clinical pathway content to a production system.

10. The method of claim 9, wherein importing content into the computerized integrated healthcare information platform includes one or more of:
importing one or more assessments;
importing one or more metrics or thresholds;
creating one or more report models; and
creating one or more pathway containers.

11. The method of claim 9, wherein configuring computerized integrated healthcare information platform components includes one or more of:
adding pathway components to a physician portal;
extending a reporting model for the physician portal; and
importing one or more of assessments and diary components to a patient application.

12. The method of claim 9, wherein deploying a test clinic includes one or more of:
creating a test clinic;
testing clinical content associated with both patients and providers; and
verifying metrics, thresholds, and reports.

13. The method of claim 9, wherein promoting clinical pathway content to a production system includes one or more of:
deploying to a production system;
adding new content to clinics; and
deploying to one or more of a patient mobile application and a patient portal.

14. A computer implemented method comprising:
providing an interface for receiving electronic patient generated healthcare data at a computerized integrated healthcare information platform from one or more of a mobile application, a patient portal, and one or more active forms, wherein the electronic patient generated healthcare data corresponds to a patient;
determining one or more scores based on the electronic patient generated healthcare data;
providing the patient generated healthcare data and the one or more scores to a physician portal;
receiving electronic treatment instruction data and clinical pathway selection from the physician portal;
generating a clinical pathway for the patient based on the electronic treatment instruction data;
receiving additional electronic patient generated healthcare data during a course of treatment based on the clinical pathway, wherein the additional electronic patient generated healthcare data corresponds to a patient;
determining a patient treatment outcome based on the additional electronic patient generated healthcare data;
continuing treatment according to the clinical pathway when the patient treatment outcome is within an acceptable range; and
providing an alert to a physician via the physician portal when the patient treatment outcome is not within an acceptable range.

15. The method of claim 14, further comprising providing an electronic alert message to at least one of the patient and the physician when a monitored parameter is not within a given range.

16. The method of claim 14, further comprising providing an electronic alert message to one or more payors that precertification is not met when patient generated healthcare data does not correspond to one or more qualifications for treatment.

17. The method of claim 14, further comprising:
identifying whether the patient needs treatment based on the patient generated healthcare data; and
routing the patient within the computerized integrated healthcare information platform to a treatment pathway based on identifying whether the patient needs treatment.

18. The method of claim 14, further comprising automatically determining a phenotype for the patient based on the patient generated healthcare data.

19. The method of claim 14, further comprising providing an interface between the computerized integrated healthcare information platform and one or more of an EMR/EHR system, a practice management system and a research and analytics system.

20. The method of claim 14, further comprising:
providing an interface for generating a custom protocol or pathway for the patient, wherein the custom protocol or pathway is received via an interface on the physician portal and is stored in a nontransitory computer readable medium.
